SEQUENCE LISTING (35 sequences)

SEQ ID NO: 1 - DNA, 68 bp, Artificial Sequence
  Synthesized
  misc_feature (30)..(35)
  misc_feature (30)..(33): n is a, c, g, or t
aatgatacggcgaccaccgagatctacacnnnnddacactctttccctacacgacgctcttccgatct

SEQ ID NO: 2 - DNA, 18 bp, Artificial Sequence
  Synthesized
  misc_feature (1)..(4)
  misc_feature (1)..(4): n is a, c, g, or t
nnnnagatcggaagagcg

SEQ ID NO: 3 - DNA, 19 bp, Artificial Sequence
  Synthetic
  misc_feature (1)..(5)
  misc_feature (1)..(5): n is a, c, g, or t
nnnnnagatcggaagagcg

SEQ ID NO: 4 - DNA, 20 bp, Artificial Sequence
  Synthetic
aatgatacggcgaccaccga

SEQ ID NO: 5 - DNA, 54 bp, Artificial Sequence
  Synthetic
  misc_feature (25)..(34)
  misc_feature (25)..(34): n is a, c, g, or t
caagcagaagacggcatacgagatnnnnnnnnnngtctcgtgggctcggagatg

SEQ ID NO: 6 - DNA, 24 bp, Homo sapiens
tttgttctttgcagtgcactttgt

SEQ ID NO: 7 - DNA, 99 bp, Artificial Sequence
  Synthesized
ctacaaggggtcgttctttaatacgactcactataggaatttctactcttgtagatttctttgcagtgcactttgttttaaaaatgtggtgatggttgc

SEQ ID NO: 8 - DNA, 17 bp, Artificial Sequence
  Synthesized
tacaaggggtcgttctt

SEQ ID NO: 9 - DNA, 17 bp, Artificial Sequence
  Synthesized
gcaaccatcaccacatt

SEQ ID NO: 10 - DNA, 24 bp, Homo sapiens
tttctgaacacagctacctgggtg

SEQ ID NO: 11 - DNA, 99 bp, Artificial Sequence
  Synthesized
ctacaaggggtcgttctttaatacgactcactataggaatttctactcttgtagattgaacacagctacctgggtgtttaaaaatgtggtgatggttgc

SEQ ID NO: 12 - DNA, 17 bp, Artificial Sequence
  Synthesized
tacaaggggtcgttctt

SEQ ID NO: 13 - DNA, 17 bp, Artificial Sequence
  Synthesized
gcaaccatcaccacatt

SEQ ID NO: 14 - DNA, 24 bp, Homo sapiens
ttttccctaacctgctgtgctttt

SEQ ID NO: 15 - DNA, 99 bp, Artificial Sequence
  Synthesized
ctacaaggggtcgttctttaatacgactcactataggaatttctactcttgtagatccctaacctgctgtgctttttttaaaaatgtggtgatggttgc

SEQ ID NO: 16 - DNA, 17 bp, Artificial Sequence
  Synthesized
tacaaggggtcgttctt

SEQ ID NO: 17 - DNA, 17 bp, Artificial Sequence
  Synthesized
gcaaccatcaccacatt

SEQ ID NO: 18 - DNA, 24 bp, Homo sapiens
tttaatctaaagaataggcgtcta

SEQ ID NO: 19 - DNA, 99 bp, Artificial Sequence
  Synthesized
ctacaaggggtcgttctttaatacgactcactataggaatttctactcttgtagatatctaaagaataggcgtctatttaaaaatgtggtgatggttgc

SEQ ID NO: 20 - DNA, 17 bp, Artificial Sequence
  Synthetic
tacaaggggtcgttctt

SEQ ID NO: 21 - DNA, 17 bp, Artificial Sequence
  Synthetic
gcaaccatcaccacatt

SEQ ID NO: 22 - DNA, 24 bp, Homo sapiens
tttcgtggatgcagtcagggccag

SEQ ID NO: 23 - DNA, 99 bp, Artificial Sequence
  Synthesized
ctacaaggggtcgttctttaatacgactcactataggaatttctactcttgtagatgtggatgcagtcagggccagtttaaaaatgtggtgatggttgc

SEQ ID NO: 24 - DNA, 17 bp, Artificial Sequence
  Synthesized
tacaaggggtcgttctt

SEQ ID NO: 25 - DNA, 17 bp, Artificial Sequence
  Synthesized
gcaaccatcaccacatt

SEQ ID NO: 26 - DNA, 24 bp, Homo sapiens
tttcacagttatgcactgcactac

SEQ ID NO: 27 - DNA, 61 bp, Artificial Sequence
  Synthesized
aaagtagtgcagtgcataactgtatctacaagagtagaaattcctatagtgagtcgtatta

SEQ ID NO: 28 - DNA, 24 bp, Homo sapiens
tttccaggttgctaaggtgatgaa

SEQ ID NO: 29 - DNA, 61 bp, Artificial Sequence
  Synthesized
aaattcatcaccttagcaacctgatctacaagagtagaaattcctatagtgagtcgtatta

SEQ ID NO: 30 - DNA, 24 bp, Homo sapiens
tttatacttgggttcgatgaagaa

SEQ ID NO: 31 - DNA, 61 bp, Artificial Sequence
  Synthesized
aaattcttcatcgaacccaagtaatctacaagagtagaaattcctatagtgagtcgtatta

SEQ ID NO: 32 - DNA, 24 bp, Homo sapiens
tttgagactaagatacatataggt

SEQ ID NO: 33 - DNA, 61 bp, Artificial Sequence
  Synthesized
aaaacctatatgtatcttagtctatctacaagagtagaaattcctatagtgagtcgtatta

SEQ ID NO: 34 - DNA, 24 bp, Homo sapiens
tttgatgtatgtttgtttcaggat

SEQ ID NO: 35 - DNA, 61 bp, Artificial Sequence
  Synthesized
aaaatcctgaaacaaacatacatatctacaagagtagaaattcctatagtgagtcgtatta

(19) United States
(12) Patent Application Publication
(10) Pub. No.: US 2022/0340966 A1
(43) Pub. Date: Oct. 27, 2022

(54) CRISPR-MEDIATED CAPTURE OF NUCLEIC ACIDS

(71) Applicant: OREGON HEALTH & SCIENCE UNIVERSITY, Portland, OR (US)

(72) Inventors: Brian J. O'ROAK, Portland, OR (US); Andrew ADEY, Portland, OR (US); Taylor MIGHELL, Portland, OR (US); Ryan MULQUEEN, Portland, OR (US); Casey THORNTON, Portland, OR (US)

(21) Appl. No.: 17/753,592

(22) PCT Filed: Sep. 9, 2020
(86) PCT No.: PCT/US2020/049966

§ 371 Date: Mar. 8, 2022

Related U.S. Application Data
(60) Provisional application No. 62/897,889, filed on Sep. 9, 2019.
(60) Provisional application No. 63/050,618, filed on Jul. 10, 2020.

(51) Int. Cl. C12Q 1/6869; C12N 15/11; C12N 9/22; C12Q 1/6806; C12N 15/10

(52) U.S. Cl. CPC C12Q 1/6869; C12N 15/111; C12N 9/22; C12Q 1/6806; C12N 15/1093; C12N 2310/20

(57) ABSTRACT

Disclosed are methods of targeted sequencing by using gRNA-endonuclease complexes and methods of designing pools of gRNAs. The disclosure also provides sequencing adapters that that comprise a double-stranded nucleic acid having a single-stranded overhang with degenerate overhanging bases. In a first aspect, methods for Dephosphorylate targeted sequencing of double-stranded nucleic acids comprises cleaving dephosphorylated double-stranded nucleic acids with a plurality of endonuclease-guide ribonucleic acid (gRNA) complexes to generate double-stranded nucleic acid fragments having phosphorylated 5′ end overhangs at targeted sites.

### RELATED APPLICATIONS

This application claims priority to U.S. Provisional Patent Application No. 62/897,889, which was filed on Sep. 9, 2019, and U.S. Provisional Patent Application No. 63/050,618, which was filed on Jul. 10, 2020, each of which is hereby incorporated by reference in its entirety.

### COPYRIGHT NOTICE

©2020 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

This disclosure relates to adapters to use for sequencing and methods for targeted sequencing of nucleic acids. More specifically, this disclosure relates to methods that include the use of endonucleases and guideRNAs, and sequencing adapters that may be used with the disclosed methods.

### BACKGROUND

CRISPR-Cas systems use Cas enzymes, which are endonucleases that form complexes with short RNA molecules (guideRNAs or gRNAs) that direct the enzyme to a specific locus in the genome via base-pairing interactions between RNA and DNA. CRISPR-Cas systems are often used to create targeted lesions in the genomes of model organisms. One particular class of Cas enzymes, Cas 12a (also known as Cpf1 or simply Cas12), has the unique property of cleaving DNA to leave 5′ single-stranded overhangs. Cas12a is directed to the target sequence by basepairing between the guide RNA and the target sequence. Cas12a catalyzes two cleavage events; the target strand can be cleaved 18 basepairs from the protospacer adjacent motif (PAM) and the non-target strand can be cleaved 23 basepairs from the PAM. The result of this reaction is two DNA molecules, each that can have a 5 basepair, 5′ overhang.

Despite reductions in genome sequencing costs, targeted sequencing methods still have high utility for research and clinical applications; e.g., screening for off-target genome editing or identifying pathogenic mutations in Mendelian disorders.

There are various sequencing approaches, for example, PCR-based targeted sequencing, using Molecular Inversion Probes (MIPs), probe-based hybridization, and using CRISPR-Cas9 mediated enrichment of target molecules. PCR-based targeted sequencing approaches rely on amplification of targeted regions from the genome followed by sequencing. These approaches require manual design and testing of primers. Also, multiplexing PCR primers often leads to errors in amplification. MIPs, also known as padlock probes, allow targeted sequencing of user-defined regions of the genome, but require a long DNA oligonucleotide (75-120 bp) for each region of DNA targeted. Further, MIP capture efficiency is affected/biased by nucleotide composition. For example, regions of high/low guanine-cytosine (GC) content perform poorly. Probe based hybridization approaches (e.g., SureSelect, SeqCap, xGen) for targeted nucleic acid enrichment are also GC content biased and require all of the probes to be biotinylated individually, which adds to synthesis costs. Commercial probe sets generated in large batches are available (typically covering the whole exome), but these are limited in their ability to support user-defined flexibility of targeted regions. A recent technology uses CRISPR-Cas9 mediated fragmentation of genomic DNA followed by size selection to enrich for on-target molecules. This method relies on a size selection step.

Current target enrichment strategies, whether PCR or hybridization based, still suffer from issues with scalability, bias, and cost (especially for custom targets). Moreover, sequencing a complete gene body remains challenging as non-exonic regions are not typically part of commercial gene panels. To address these challenges, disclosed herein are sequencing adapters and methods that enable efficient and uniform capture of any set of genomic loci.

### BRIEF DESCRIPTION OF THE DRAWINGS

FIG. 1 is a graphical overview of one embodiment of the methods disclosed herein.

FIG. 2 shows graphical representations of four examples (each panel a, b, c, d being an example) of adapters that have been designed for a test target sequence.

FIG. 3 is a graphical representation of the results from a pilot guide experiment. Panel a is a histogram of position of first base of read 1 in relation to the end of PAM (i.e. the start of the protospacer). Reads originating from the Cas12a proximal and distal molecules are colored differently. Panel b is a graph of the ratio of Cas12a distal to proximal reads for all guides, rank ordered by magnitude of ratio. Panel c is a graph of coverage versus vases downstream of cut site. This is coverage of bases, from read 1, as a function of distance downstream from nearest cut site. Panel d is a graph of coverage versus vases downstream of cut site. This is coverage of bases, from read 2, as a function of distance downstream from nearest cut site.

FIG. 4 shows graphical representations of the results from a pilot guide experiment using an embodiment of the methods disclosed herein. Panel a is a graph of reads versus guides, representing read uniformity for guides in the pilot experiment. Dashed lines indicate a log10 window within which 49.3% of guides performed. Panel b is a graph of features versus feature coefficients. The twenty features in the linear regression model with the largest positive and negative coefficients are shown. Panel c is a graph of observed log reads versus predicted log reads, representing performance of the linear regression model on fully withheld test data. Panel d is a heatmap representing feature coefficients of individual position-specific nucleotides.

FIG. 5 shows graphical representations of the results from trained models. Panel a is a graph of spearman correlation (predicted vs. observed) versus features used. Models were iteratively trained with more features, successively adding features with the highest absolute value coefficient. Panel b is a graph of spearman correlation versus training set size. Models were trained with varying training set sizes.

FIG. 6A is a graph of reads versus guides, showing read uniformity for guides in the optimized experiment. Dashed lines indicate a log10 window within which 54.0% of guides performed.

FIG. 6B is a graph of coverage versus bases, showing per-base read coverage across the full target with downsampled datasets.

FIG. 6C is a graph of coverage versus GC content, representing coverage of bases within different 100 basepair GC content bins.

FIG. 6D is a graphical representation of precision and recall for single nucleotide variant calling of NA12878 compared to the “Platinum” variant calls.

FIG. 7 shows graphical representations of the results from the optimized guide set experiment selected using the machine learning model. Panel a is a graph of observed log reads versus predicted log reads. This is predicted versus observed performance (as defined by assigned reads) for the optimized guide set. Pearson r=0.39. Panel b is a graph of coverage versus bases, representing coverage uniformity for all bases outside of repeats (as defined by Repeat Masker) for various downsampled datasets. Panel c is a graph representing precision and recall for single nucleotide variants called outside of repeats (as defined by Repeat Masker) at different downsampled read pairs.

FIG. 8 is a schematic of the method as applied to massively parallel sequencing (panels A, B, C, D).

### DETAILED DESCRIPTION

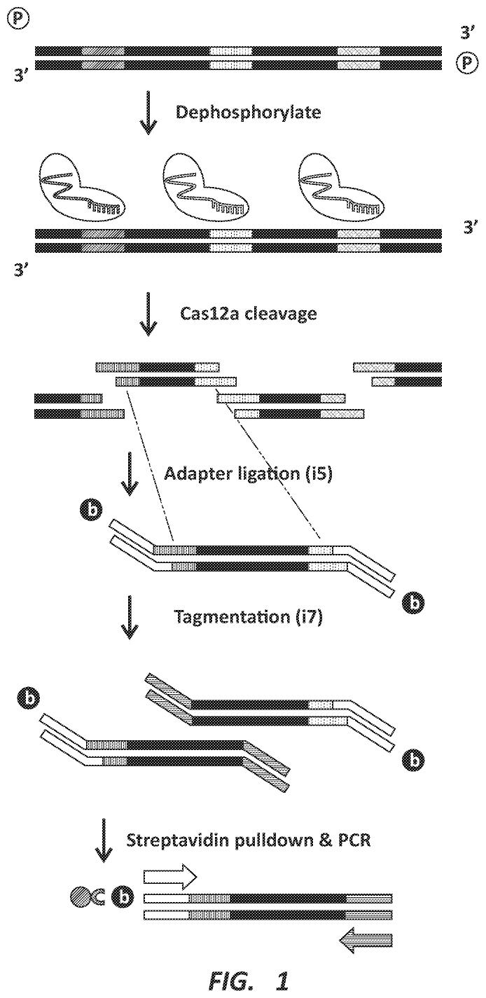

Cas12a-mediated genomic fragmentation, mediated by a pool of targeted gRNAs, results in enrichment of ligatable overhanging ends at targeted loci. Moreover, Cas12a cleavage can occur completely in vitro on naked DNA. Specific gRNAs can be generated in bulk at low cost by synthesizing pools of DNA oligonucleotides containing the gRNA sequence as well as the T7 RNA polymerase priming site. In vitro transcription can then be used to generate pools of functional gRNAs. In order to reduce spurious ligation events, genomic DNA can be enzymatically dephosphorylated prior to incubation with the Cas12a-gRNA RNP (FIG. 1). Cas12a cleavage can result in a 5′ overhang of four to five nucleotides. Therefore, custom biotinylated adapters containing the IIlumina i5 flow cell and priming sequences, as well as overhangs of four or five degenerate nucleotides (Table 1) were designed. Following ligation of the i5 adapter, tagmentation with Tn5 transposase can add the i7 sequencing adapter. Finally, to enrich for molecules with a ligated i5 adapter (and deplete molecules with two i7 adapters), a streptavidin-mediated pulldown can be performed, followed by polymerase chain reaction (PCR) directly on the streptavidin beads (FIG. 1). In FIG. 1, “P” denotes phosphorylation and “b” denotes biotin.

TABLE 1

| Primers and | Oligonucleotides |
|---|---|
| i5 adapter top | /5′biotin/AATGATACGGCGACCACCGAGATCTACACNNNNDD |
| | ACACTCTTTCCCTACACGACGCTCTTCCGATCT (SEQ ID |
| | NO: 1) |
| i5 adapter | NNNNAGATCGGAAGAGCG (SEQ ID NO: 2) |
| bottom 4N | |
| i5 adapter | NNNNNAGATCGGAAGAGCG (SEQ ID NO: 3) |
| bottom 5N | |
| i5 enrichment | AATGATACGGCGACCACCGA (SEQ ID NO: 4) |
| primer | |
| i7 enrichment | CAAGCAGAAGACGGCATACGAGAT[10bp_index]GTCTCGTG |
| primer (Nextera) | GGCTCGGAGATG (SEQ ID NO: 5) |

Disclosed herein are methods for targeted sequencing of double-stranded nucleic acids. In a first aspect, methods for targeted sequencing of double-stranded nucleic acids comprises cleaving dephosphorylated double-stranded nucleic acids with a plurality of endonuclease-guide ribonucleic acid (gRNA) complexes to generate double-stranded nucleic acid fragments having phosphorylated 5′ end overhangs at targeted sites. The methods further comprise ligating a first adapter to the targeted sites of the double-stranded nucleic acid fragments and fragmenting further the double-stranded nucleic acids fragments at random sites. The methods of the first aspect also comprise adding a second adapter at the random sites and amplifying selectively nucleic acid sequences containing the first adapter and the second adapter to generate a library of target sequences. The first adapter and second adapter each comprise priming sites.

Any naturally-occurring or synthetic endonuclease that is guided and cleaves double-stranded nucleic acids and leaves a 5′ overhang (i.e., a “sticky end”) may be used. The endonuclease may be CRISPR-Cas12a. In a first embodiment of the method of the first aspect, the plurality of endonuclease-gRNA complexes are ribonucleoproteins. The endonuclease-gRNA complexes may comprise CRISPR-Cas12a-based endonuclease complexed with one of a plurality of different gRNA to provide a plurality of different endonuclease-gRNA complexes. “Cas12a-based” as used herein encompasses any Cas12a from different species and any modified Cas12a that retains overhang functionality (i.e., generates overhangs or “sticky ends” instead of blunt ends). Herein, gRNA may be targeted to the target sequence and may comprise a protospacer adjacent motif compatible with the CRISPR-Cas12a-based endonuclease.

The first embodiment of the method of the first aspect may further comprise synthesizing double-stranded nucleic acids encoding the different gRNA sequences and transcribing the synthesized double-stranded nucleic acids in vitro into the gRNAs. The method can further include complexing the gRNA with the CRISPR-Cas12a-based endonuclease to form the plurality of different endonuclease-gRNA complexes. Commercially-available RNAs may be used to complex the CRISPR-Cas12a-based endonuclease to form the plurality of different endonuclease-gRNA complexes.

In the methods of the first aspect, the double-stranded nucleic acids may comprise deoxynucleic acids (DNA), including naturally-occurring DNA, but not limited to genomic DNA, mitochondrial DNA, and cell-free DNA. The double-stranded nucleic acids may comprise synthetic DNA, but not limited to complementary DNA (cDNA) (including as reverse transcribed from RNA), and polymerase chain reaction (PCR) products. The methods of the first aspect may further comprise dephosphorylating double-stranded nucleic acids to provide the dephosphorylated double-stranded nucleic acids. Optionally, the methods may further comprise, prior to dephosphorylation, removing existing 5′ end overhangs from double-stranded nucleic acids to provide the double-stranded nucleic acids for dephosphorylation.

In other embodiments of the methods of the first aspect, the first adapter comprises double-stranded nucleic acids that comprise degenerate overhanging bases compatible with the phosphorylated 5′ end overhangs of the double-stranded nucleic acid fragments. The first adapter may further comprise a unique molecular identifier, index sequence, or both. For example, a plurality of the first adapters are present in a mixture with numerous different unique molecular identifiers. Further, in the methods of the first aspect, a pulldown reaction targeted to the first adapter may be used to pulldown products. The first adapter may comprise a 5′ biotin modification compatible with streptavidin pulldown, a digoxigen (DIG) modification compatible with DIG antibody pulldown, a chemical modification compatible with isolation via click chemistry reaction with an alkyne or azide solid resin, or a poly-histidine tag modification compatible with nickel-containing solid resin pulldown. Yet in another embodiment, the method of the first aspect may further comprise enriching the double-stranded nucleic acid fragments containing the first adapter ligated thereto, preceding or after fragmenting further the double-stranded nucleic acids.

In the method of the first aspect, fragmenting further the double-stranded nucleic acids fragments at random sites and adding the second adapter may comprise using a transposase with a commercially-available or custom adapter. It may be possible to accomplish fragmenting the double-stranded nucleic acid fragments at random sites and adding the second adapter at the random sites in a single step or in two or more steps. Enzymatic fragmentation, sonic fragmentation, or mechanical shearing may be used to further fragment the double-stranded nucleic acids at random sites.

To generate a library of target sequences, amplifying selectively nucleic acid sequences containing the first adapter and the second adapter to generate a library of target sequences may comprise a pulldown reaction targeted to the first adapter to generate pulldown products and then amplifying the products to generate the library of target sequences. The methods of the first aspect may further comprise generating the library of target sequences without a size selection step prior to addition of the first and second adapters. Library quantification techniques, size selection, massive parallel sequencing, informatic protocols, or combinations thereof, to the library of target sequences may also be performed. In methods disclosed herein, target sequences may comprise whole genes, a region of interest, or a list of regions of interest. The target sequences may comprise regions of high or low guanine-cytosine (GC) content.

Also disclosed herein are methods of designing a pool of guide RNA (gRNA) to be complexed with an endonuclease. In a second aspect, methods of designing a pool of gRNA to be complexed with an endonuclease comprise identifying all possible target sites of the endonuclease within target sequences, providing a first plurality of gRNA to target each of the identified possible target sites of the endonuclease, and complexing each of the first plurality of gRNAs with the endonuclease to form a first plurality of endonuclease-gRNA complexes. The method of the second aspect further includes performing the steps of the methods of the first aspect utilizing the first plurality of endonuclease-gRNA complexes to generate a first library of the target sequences, and includes comparing the first library of the target sequences to a known library of the target sequences. Yet further, the methods comprise determining a subset of the first plurality of endonuclease-gRNA complexes that generate target sequences aligned with the known library of the target sequences, determining molecular features of the target sequences associated with the subset of the first plurality of endonuclease-gRNA complexes, and designing a second plurality of gRNA to the same or additional target sequences that also have the molecular features associated with performance of the subset of the first plurality of endonuclease-gRNA complexes. Determining the molecular features of the target sequences associated with the subset of the first plurality of endonuclease-gRNA complexes can utilize machine learning techniques.

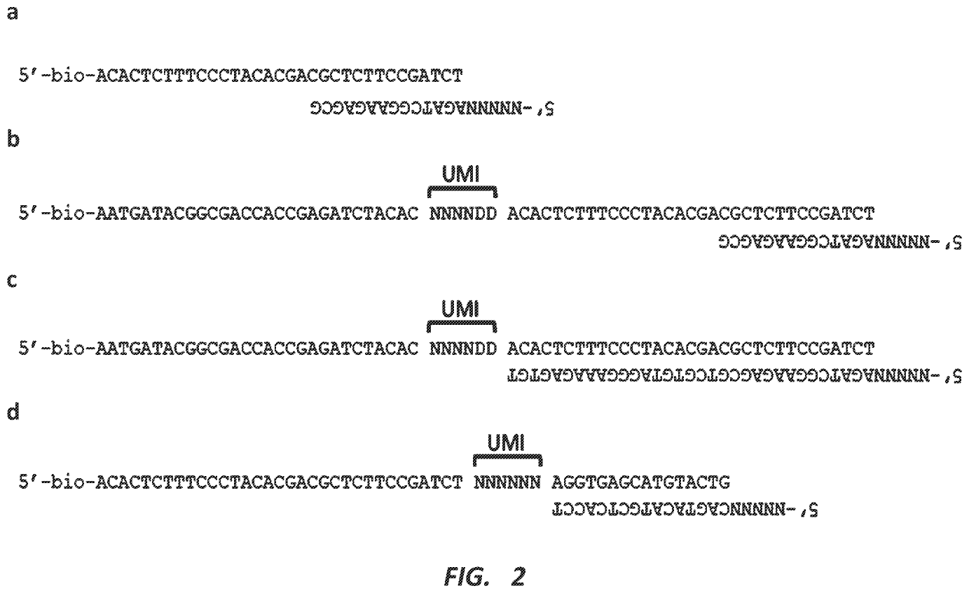

Sequencing adapter mixtures are also disclosed herein. In a third aspect, a first sequencing adapter mixture comprises double-stranded nucleic acids each having a first strand and a second strand, wherein each first strand comprises priming sites and optionally, a unique molecular identifier, index sequence, or both (FIG. 2). Unique molecular identifiers are degenerate bases that are unique to each molecule. Sequencing adapter mixtures may comprise a plurality of different double-stranded nucleic acids each having a first strand and a second strand, wherein each first strand comprises priming sites and a unique molecular identifier. Each second strand in the double-stranded nucleic acids is complementary to the respective first strand but contains a 5′ overhang of one, two, three, four, or five degenerate bases. Each second strand in the double-stranded nucleic acids forms a double-stranded region with the first strand. The double-stranded region may not extend along the entire length of the first strand, depending on the length of the second strand; however, the second strand always has a 5′ overhang of degenerate bases. In the first sequencing adapter of the third aspect, the unique molecular identifier, index sequence, or both may be located towards the 5′ end of the first strand when compared to sequences complementary to the respective second strand. The first sequencing adapter of the third aspect may further comprise a 5′ biotin modification compatible with streptavidin pulldown, a digoxigen (DIG) modification compatible with DIG antibody pulldown, a chemical modification compatible with isolation via click chemistry reaction with an alkyne or azide solid resin, or a poly-histidine tag modification compatible with nickel-containing solid resin pulldown.

Adapters disclosed herein can be compatible with massively parallel sequencing of the IIlumina platforms. Each adapter consists of two annealed oligos: one strand is biotinylated (red “bio”) and the other strand is the “splint”, containing degenerate overhanging bases, which promotes ligation. As examples, the adapter in FIG. 2 panel A includes the partial IIlumina i5 sequencing adapter and is compatible with an i5 index. The adapter in FIG. 2 panel B contains the entire i5 sequencing adapter. The adapter has a unique molecular identifier (UMI) instead of an i5 index. The adapter in FIG. 2 panel C is the same as in FIG. 2 panel B, except with a longer splint. The adapter in FIG. 2 panel D includes the partial IIlumina i5 adapter. This adapter has a UMI that is read at the beginning of read 1 (instead of in the index read, as in FIG. 2 panel B and FIG. 2 panel C. Red ‘bio’ indicates biotinylation.

### EXAMPLES

### Example 1—Design of Pilot Guide Set

A pilot set of guides was designed targeting 47 known and candidate risk genes for Joubert Syndrome (JS, Table 2), representing 3.5 megabases of DNA. RefSeq hg19 genomic coordinates were obtained for the 47 genes from UCSC Table Browser as a bed file. Overlapping intervals were merged with Galaxy to obtain a single interval per gene, to which were then padded with 3,000 basepairs upstream and 500 basepairs downstream, in hopes of capturing promoters and 3′ untranslated region sequences. Then, FlashFry16 was used to find all possible Cas12a target sites (i.e. the presence of “TTTN” PAM) within these target regions and to report the copy number of each potential gRNA target sequence. Filtered out were the guide target sequences that had copy number greater than one, or that had many similar off target sequences (>25 off targets within 1 edit distance, or >100 off targets within 2 edit distance). Also filtered were guides that overlapped a common single nucleotide polymorphism (SNP, minor allele fraction >0.1%, dbSNP, release 151. Then, for each gene, targets were defined by simply enumerating 500 basepair intervals, and the gRNA with cut site closest to the target were selected. This resulted in 7,176 guide sequences. Then DNA oligo sequences that contained the following in the 5′ to 3′ direction were designed: dial out PCR priming site, T7 RNA polymerase priming site, crRNA backbone (including Acidaminococcus sp. BV3L6 (As) Cas12a constant loop region), protospacer sequence, Dral cut-site (“TTTAAA”), and another dial out PCR priming site (select examples shown in Table 3). These gRNA templates were synthesized as 99-mers on 12,000-feature oligo chips (CustomArray).

TABLE 2
Genomic coordinates for 47 known and candidate
risk genes for Joubert Syndrome

| Chromosome | Start | End | Gene |
|---|---|---|---|
| chr1 | 3728144 | 3776797 | CEP104 |
| chr1 | 5922367 | 6055533 | NPHP4 |
| chr1 | 200609434 | 200642126 | DDX59 |
| chr1 | 231467976 | 231476618 | EXOC8 |
| chr10 | 97422652 | 97456900 | TCTN3 |
| chr10 | 104260718 | 104393714 | SUFU |
| chr10 | 104432983 | 104477190 | ARL3 |
| chr11 | 61126472 | 61137477 | TMEM138 |
| chr11 | 61156831 | 61166835 | TMEM216 |
| chr11 | 73723258 | 73885064 | C2CD3 |
| chr11 | 124963765 | 124984659 | TMEM218 |
| chr11 | 125750508 | 125771041 | HYLS1 |
| chr12 | 88442289 | 88538993 | CEP290 |
| chr12 | 94699668 | 94856764 | CEP83 |
| chr12 | 111048831 | 111087435 | TCTN1 |
| chr12 | 124152659 | 124193450 | TCTN2 |
| chr13 | 73353229 | 73591092 | PIBF1 |
| chr14 | 45428392 | 45544134 | TOGARAM1 |
| chr14 | 58891102 | 59016049 | KIAA0586 |
| chr15 | 90170700 | 90201682 | KIF7 |
| chr16 | 3547930 | 3589548 | CLUAP1 |
| chr16 | 27558467 | 27792192 | KIAA0556 |
| chr16 | 53632650 | 53740850 | RPGRIP1L |
| chr16 | 75571514 | 75593184 | TMEM231 |
| chr17 | 6481144 | 6547247 | KIAA0753 |
| chr17 | 8075795 | 8082714 | TMEM107 |
| chr17 | 19238184 | 19284506 | B9D1 |
| chr17 | 56282296 | 56299966 | MKS1 |
| chr19 | 41859821 | 41873078 | B9D2 |
| chr2 | 27666739 | 27715678 | IFT172 |
| chr2 | 110880412 | 110965639 | NPHP1 |
| chr2 | 202484406 | 202511252 | TMEM237 |
| chr2 | 232060259 | 232241090 | ARMC9 |
| chr2 | 232596634 | 232649037 | PDE6D |
| chr22 | 39049657 | 39070355 | CBY1 |
| chr3 | 56588183 | 56656364 | CCDC66 |
| chr3 | 93695982 | 93775022 | ARL13B |
| chr3 | 132398952 | 132444303 | NPHP3 |
| chr4 | 15468488 | 15603680 | CC2D2A |
| chr5 | 37105829 | 37252530 | CPLANE1 |
| chr5 | 122680078 | 122762286 | CEP120 |
| chr6 | 135604609 | 135821903 | AHI1 |
| chr7 | 130033111 | 130084051 | CEP41 |
| chr8 | 67973602 | 68109349 | CSPP1 |
| chr8 | 94764071 | 94831962 | TMEM67 |
| chr9 | 139322566 | 139337274 | INPP5E |
| chrX | 13749831 | 13787980 | OFD1 |

TABLE 3
Examples of designed oligo sequences

| oligo_id | contig | start | stop | target | full_synth_oligo | orientation | pool | dial_out_F | dial_out_R |
|---|---|---|---|---|---|---|---|---|---|
| 1 | chr12: | 101487 | 101511 | TTTGTTCTTTG | CTACAAGGGGTCGTTCTTTAATACG | RVS | JS_pool_ | TACAAGG | GCAACCAT |
| | 94699668- | | | CAGTGCACTTT | ACTCACTATAGGAATTTCTACTCTT | | 1 | GGTCGTT | CACCACAT |
| | 94856764 | | | GT | GTAGATTTCTTTGCAGTGCACTTTG | | | CTT | T |
| | | | | (SEQ ID NO: 6) | TTTTAAAAATGTGGTGATGGTTGC | | | (SEQ ID | (SEQ ID |
| | | | | | (SEQ ID NO: 7) | | | NO: 8) | NO: 9) |
| 2 | chr2: | 49469 | 49493 | TTTCTGAACAC | CTACAAGGGGTCGTTCTTTAATACG | RVS | JS_pool_ | TACAAGG | GCAACCAT |
| | 232060259- | | | AGCTACCTGG | ACTCACTATAGGAATTTCTACTCTT | | 1 | GGTCGTT | CACCACAT |
| | 232241090 | | | GTG | GTAGATTGAACACAGCTACCTGGGT | | | CTT | T |
| | | | | (SEQ ID NO: 10) | GTTTAAAAATGTGGTGATGGTTGC | | | (SEQ ID | (SEQ ID |
| | | | | | (SEQ ID NO: 11) | | | NO: 12) | NO: 13) |
| 3 | chr16: | 87490 | 87514 | TTTTCCCTAAC | CTACAAGGGGTCGTTCTTTAATACG | RVS | JS_pool_ | TACAAGG | GCAACCAT |
| | 53632650- | | | CTGCTGTGCTT | ACTCACTATAGGAATTTCTACTCTT | | 1 | GGTCGTT | CACCACAT |
| | 53740850 | | | TT | GTAGATCCCTAACCTGCTGTGCTTT | | | CTT | T |
| | | | | (SEQ ID NO: 14) | TTTTAAAAATGTGGTGATGGTTGC | | | (SEQ ID | (SEQ ID |
| | | | | | (SEQ ID NO: 15) | | | NO: 16) | NO: 17) |
| 4 | chr3: | 51495 | 51519 | TTTAATCTAAA | CTACAAGGGGTCGTTCTTTAATACG | RVS | JS_pool_ | TACAAGG | GCAACCAT |
| | 93695982- | | | GAATAGGCGT | ACTCACTATAGGAATTTCTACTCTT | | 1 | GGTCGTT | CACCACAT |
| | 93775022 | | | CTA | GTAGATATCTAAAGAATAGGCGTCT | | | CTT | T |
| | | | | (SEQ ID NO: 18) | ATTTAAAAATGTGGTGATGGTTGC | | | (SEQ ID | (SEQ ID |
| | | | | | (SEQ ID NO: 19) | | | NO: 20) | NO: 21) |
| 5 | chr1: | 57384 | 57408 | TTTCGTGGATG | CTACAAGGGGTCGTTCTTTAATACG | FWD | JS_pool_ | TACAAGG | GCAACCAT |
| | 5922367- | | | CAGTCAGGGC | ACTCACTATAGGAATTTCTACTCTT | | 1 | GGTCGTT | CACCACAT |
| | 6055533 | | | CAG | GTAGATGTGGATGCAGTCAGGGCC | | | CTT | T |
| | | | | (SEQ ID NO: 22) | AGTTTAAAAATGTGGTGATGGTTGC | | | (SEQ ID | (SEQ ID |
| | | | | | (SEQ ID NO: 23) | | | NO: 24) | NO: 25) |

### Example 2—Guide Amplification and In Vitro Transcription of Pilot Guide Set

PCR was used to amplify the gRNA templates from the oligo pool using dial out primers. Reactions contained 1×KAPA HiFi Hotstart Readymix, 10 ng of template, 0.5 μM primers, and 1×SYBR Green. Reactions were pulled upon completing exponential amplification, which occurred at 19-22 cycles. Agarose gel electrophoresis confirmed bands of 99 basepairs. Reactions were purified with NucleoSpin PCR cleanup columns (Machery Nagel). Then, purified products were treated with Dral restriction enzyme in order to remove the priming site downstream of the gRNA sequence. Reactions contained 500 ng of PCR product, 40 units of Dral (New England BioLabs), and 1× CutSmart buffer. Incubation was done at 37° and proceeded overnight. Reactions were cleaned up with NucleoSpin PCR cleanup columns, and complete digestion was confirmed with agarose gel electrophoresis.

MEGAscript T7 Transcription Kit (Thermo Fisher Scientific) was used to generate gRNAs from the templates. Reactions contained ˜60-130 ng DNA (depending on recovery from previous step) and were incubated at 37° overnight. Following incubation, reactions were treated with TURBO DNase and incubated at 37° for 15 minutes. Then, RNA Clean & Concentrator (Zymo Research) columns were used to purify RNA. RNA was quantified with Qubit RNA Broad Range Assay (Thermo Fisher Scientific) and diluted to 10 μM.

### Example 3—Sequencing

Briefly, genomic DNA was treated with phosphatase to enzymatically remove the terminal phosphates from genomic DNA molecules. Then, genomic DNA was treated with gRNA-complexed Cas12a, which created overhangs specifically at targeted sites. Custom i5 adapters that contained complementary overhangs, a unique molecular identifier (UMI), and 5′ biotin modification were added with T4 ligase. Then, the i7 adapter was added through Tn5 tagmentation. A streptavidin-mediated pulldown step purifies those molecules that have an i5 adapter (excluding the molecules with only i7 adapters), and on-bead PCR (followed by size selection/purification as necessary) generated ready-to-sequence libraries. All libraries were sequenced in paired-end mode on the IIlumina NextSeq500 platform with Mid Output 150 cycle v2.5 kits. Cycles were allocated as follows: 35 cycles for read 1, 10 cycles for index 1, 6 or 10 cycles for index 2 (depending on the presence of unrelated multiplexed libraries), and 113 or 118 cycles for read 2.

### Example 4—Sequencing Data Processing and Analysis

The custom adapter contained a six nucleotide unique molecular identifier (UMI) in place of the i5 index. The first step of an informatics pipeline was appending the sequence from the i5 index read to the end of the read name line of both read 1 and 2 fastq files with a custom python script. This was done for compatibility with UMI-tools17. Next, adapters were trimmed with cutadapt and paired end reads were aligned to the hg19 reference genome with BWA-MEM. Following paired end read alignment, duplicates were removed with UMI-tools dedup.

### Example 5—Analysis of Pilot Guide Set Performance

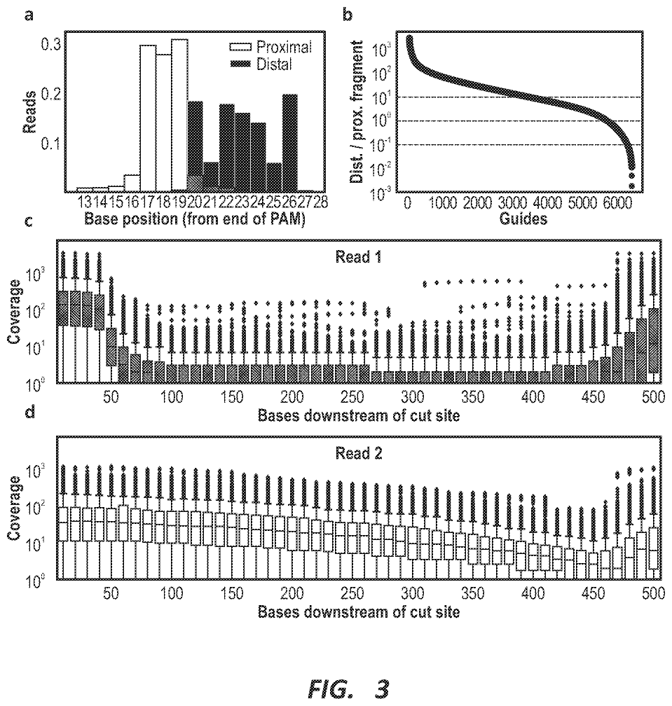

Combined paired-end sequencing data from several pilot guide set libraries prepared from the well-studied CEPH/Hapmap sample NA12878 resulted in 5.9% of reads on target, corresponding to a 52.4-fold enrichment. As a primary error modality of array synthesis is single base deletions, a predicted off target list was generated by aggregating all sites in the genome at which gRNAs with a single base deletion aligned (495,299 sites). 12.7% of sequencing reads aligned to these predicted off target sites, which is significantly more than aligned to the same number of size-matched random genomic intervals (1.75%, p<0.01, Chi-squared test). Since Cas12a cleavage results in symmetrical 5′ overhangs, approximately equal numbers of reads were expected to result from ligation to both overhangs. However, this was not the case: 56% of guides had greater than 10 times more reads aligning to the enzyme-distal overhang (FIG. 3). This bias may be due to Cas12a remaining bound to the enzyme proximal fragment and sterically inhibiting ligation, though treatment with sodium dodecyl sulfate after cleavage did not reduce the bias.

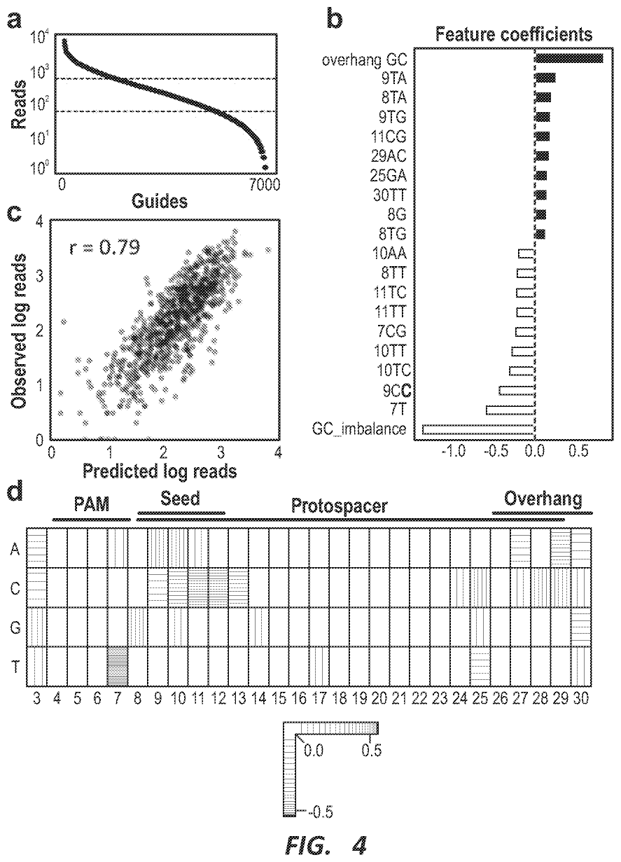

Inspection of the read alignments revealed accumulation of the first read at programmed cut sites, consistent with ligation of the i5 adapter directly to the cut site overhang. In contrast, the second read was scattered across the inter-guide interval, consistent with this adapter being appended by semi-random tagmentation (FIG. 3 panels c and d). The first read of 92.6% of on-target read pairs began within 5 bases of a predicted guide cut site. Additionally, the starting position of the first read corresponded to the expected cut sites of Cas12a (i.e. after the 18th and 23rd bases downstream of the PAM, FIG. 3 panel a). The number of reads assigned to each guide was used as a proxy for the performance of that guide. Comparing the performance of capture across the full guide set revealed a thousand-fold difference between the best and worst performing guides; however, 49.3% of guides performed within one log10 difference (FIG. 4 panel a).

### Example 6—Modeling Sequence Determinants of Guide Performance

The performance of guides was estimated by the number of sequencing reads that aligned to the predicted cut site. Namely, a read was assigned to a guide if the first base of the read was within the 16th to 26th position downstream of a guide's PAM. An additional pseudocount read was added to all guide counts, enabling log transformation of all read counts, which were used as the dependent variable. 667 sequence-based features were collected as in previous work modeling Cas12a in vivo activity. Four bases upstream of the PAM and six bases downstream of the protospacer were considered. Position-specific nucleotides and dinucleotides were included (excluding the first three positions of the PAM, which are fixed as “T”), as well as two features relating to GC content: the GC imbalance of the protospacer (i.e. how far the actual GC content was from 50%), and the GC content of the predicted overhang (positions 26-30). Additionally, the estimated minimum free energy of the RNA molecule were included.

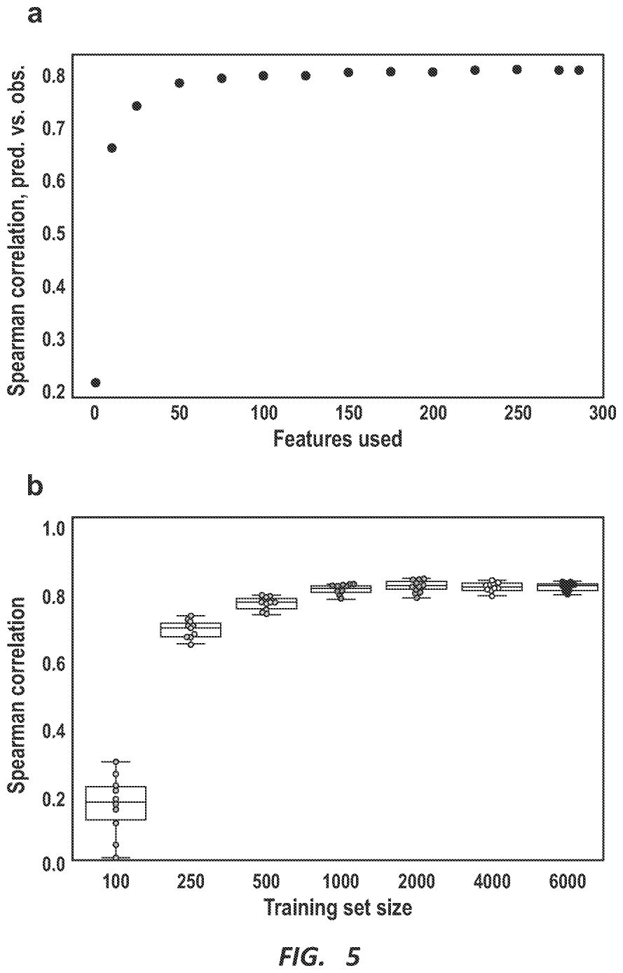

Feature selection was done with the elastic net procedure, implemented in scikit-learn version 0.19.0. Optimal hyperparameters was found with cross validation (ElasticNetCV) on 90% of the data (6,447 guides). This procedure resulted in 287 features with non-zero coefficients. To further eliminate inconsequential features, ordinary least squares linear regression models were trained with increasing numbers of features (rank ordered by elastic net coefficient absolute value) and made predictions on the 10% (729) fully withheld guides. Prediction performance did not substantially improve once the top ˜100 features were added (FIG. 5 panel a). Therefore, a final ordinary least squares linear regression model was fit to all available data (training and test), with the 100 selected features, which then were used to make predictions for the optimized guide set.

Hyperparameters were chosen with nested cross-validation, and the resulting model on fully withheld data was tested. The predicted and observed scores were highly correlated (Pearson r=0.79, FIG. 4 panel c). Overall, 287 features were assigned non-zero coefficients, and based on a plateau in predictive performance the top 100 (FIG. 5 panel a) were used. Consistent with previous work, a thymine at the fourth position of the PAM is strongly disfavored. Other important features were related to GC content; GC imbalance of the guide was strongly disfavored, while high GC content at the overhang positively related to performance, likely due to increased ligation efficiency (FIG. 4 panel b). Inspection of contributions from single position-specific nucleotides suggests the most important positions are within the seed and overhang regions (FIG. 4 panel d).

### Example 6—Design and Analysis of Optimized Guide Set

The same procedure as for the pilot guide set was used for obtaining padded genomic coordinates, identifying all possible Cas12a target sites, and excluded potential guides with copy number >1 and overlapping SNPs >0.001 allele frequency (dbSNP build 153). A restricted list of 34 genes, representing high confidence JS risk genes (Table 4), was used. Also implemented was a more sophisticated procedure for picking guides. First, two guide sets were designed, one targeting the forward genomic strand and one targeting the reverse genomic strand, such that consecutive guides alternated orientation. After picking a guide, the next target was defined as 250 basepairs downstream of the predicted cut site. A set of criteria was established, prioritizing high-scoring guides, guides most proximal to the target, and guides with a low number of predicted off target sites. Predicted off target sites for each guide were found by enumerating all possible single nucleotide deletions from the guide sequence and finding perfect matches for these in the genome. If there were no guides of the correct orientation fulfilling the criteria and within 250 basepairs of the target, the search was broadened to guides in the opposite orientation. If there were still no suitable guides, no guides were chosen at this step. Once this process had been completed for all genes, all “gaps” (i.e. no guides present) of greater than 600 basepairs were identified. The reasoning was that flanking the gaps with guides in the optimal orientation (i.e. forward guides upstream and reverse guides downstream of the gap) may maximize the ability to obtain coverage in the gap regions. So, if correctly oriented guides were present within 100 basepairs of the gap, regardless of predicted performance, those guides were additionally picked. A total of 11,438 guides were picked for the optimized set, and guides were synthesized as two oPools at the picomole per oligo scale (Integrated DNA Technologies, select examples shown in Table 5).

TABLE 4
A restricted gene list representing high
confidence JS risk genes

| Chromosome | Start | End | Gene |
|---|---|---|---|
| chr6 | 135604609 | 135821903 | AHI1 |
| chr3 | 93695982 | 93775022 | ARL13B |
| chr2 | 232060259 | 232241090 | ARMC9 |
| chr17 | 19238184 | 19284506 | B9D1 |
| chr11 | 73723258 | 73885064 | C2CD3 |
| chr4 | 15468488 | 15603680 | CC2D2A |
| chr3 | 56588183 | 56656364 | CCDC66 |
| chr1 | 3728144 | 3776797 | CEP104 |
| chr5 | 122680078 | 122762286 | CEP120 |
| chr12 | 88442289 | 88538993 | CEP290 |
| chr12 | 94699668 | 94856764 | CEP83 |
| chr5 | 37105829 | 37252530 | CPLANE1 |
| chr8 | 67973602 | 68109349 | CSPP1 |
| chr2 | 27666739 | 27715678 | IFT172 |
| chr9 | 139322566 | 139337274 | INPP5E |
| chr16 | 27558467 | 27792192 | KIAA0556 |
| chr14 | 58891102 | 59016049 | KIAA0586 |
| chr17 | 6481144 | 6547247 | KIAA0753 |
| chr15 | 90170700 | 90201682 | KIF7 |
| chr17 | 56282296 | 56299966 | MKS1 |
| chr2 | 110880412 | 110965639 | NPHP1 |
| chr1 | 5922367 | 6055533 | NPHP4 |
| chrX | 13749831 | 13787980 | OFD1 |
| chr13 | 73353229 | 73591092 | PIBF1 |
| chr16 | 53632650 | 53740850 | RPGRIP1L |
| chr12 | 111048831 | 111087435 | TCTN1 |
| chr12 | 124152659 | 124193450 | TCTN2 |
| chr10 | 97422652 | 97456900 | TCTN3 |
| chr11 | 61126472 | 61137477 | TMEM138 |
| chr11 | 61156831 | 61166835 | TMEM216 |
| chr16 | 75571514 | 75593184 | TMEM231 |
| chr2 | 202484406 | 202511252 | TMEM237 |
| chr8 | 94764071 | 94831962 | TMEM67 |
| chr14 | 45428392 | 45544134 | TOGARAM1 |

TABLE 5
Select examples of guides that were picked from optimized set

| oligo_ | | | | | | |
|---|---|---|---|---|---|---|
| id | contig | start | stop | target | full_synth_oligo | orientation |
| 1 | chr3: | 31 | 55 | TTTCACAGTTATG | AAAGTAGTGCAGTGCATAACTGTATC | RVS |
| | 93695982- | | | CACTGCACTAC | TACAAGAGTAGAAATTCCTATAGTGA | |
| | 93775022 | | | (SEQ ID NO: 26) | GTCGTATTA (SEQ ID NO: 27) | |
| 2 | chr3: | 489 | 513 | TTTCCAGGTTGCT | AAATTCATCACCTTAGCAACCTGATCT | RVS |
| | 93695982- | | | AAGGTGATGAA | ACAAGAGTAGAAATTCCTATAGTGA | |
| | 93775022 | | | (SEQ ID NO: 28) | GTCGTATTA (SEQ ID NO: 29) | |
| 3 | chr3: | 992 | 1016 | TTTATACTTGGGT | AAATTCTTCATCGAACCCAAGTAATC | RVS |
| | 93695982- | | | TCGATGAAGAA | TACAAGAGTAGAAATTCCTATAGTGA | |
| | 93775022 | | | (SEQ ID NO: 30) | GTCGTATTA (SEQ ID NO: 31) | |
| 4 | chr3: | 1603 | 1627 | TTTGAGACTAAG | AAAACCTATATGTATCTTAGTCTATCT | RVS |
| | 93695982- | | | ATACATATAGGT | ACAAGAGTAGAAATTCCTATAGTGA | |
| | 93775022 | | | (SEQ ID NO: 32) | GTCGTATTA (SEQ ID NO: 33) | |
| 5 | chr3: | 1914 | 1938 | TTTGATGTATGTT | AAAATCCTGAAACAAACATACATATC | RVS |
| | 93695982- | | | TGTTTCAGGAT | TACAAGAGTAGAAATTCCTATAGTGA | |
| | 93775022 | | | (SEQ ID NO: 34) | GTCGTATTA (SEQ ID NO: 35) | |

MEGAscript T7 Transcription Kit was used to generate gRNAs directly from the single-stranded templates. The recommended reaction volumes were scaled-up five-fold, and 50 picomoles of oPool template as well as 50 picomoles of T7 promoter were added. Reactions were incubated at 37° overnight. Following incubation, reactions were treated with TURBO DNase and incubated at 37° for 15 minutes. Then, RNA Clean & Concentrator (Zymo Research) columns were used to purify RNA. RNA was quantified with Qubit RNA Broad Range Assay (Thermo Fisher Scientific) and diluted to 10 μM.

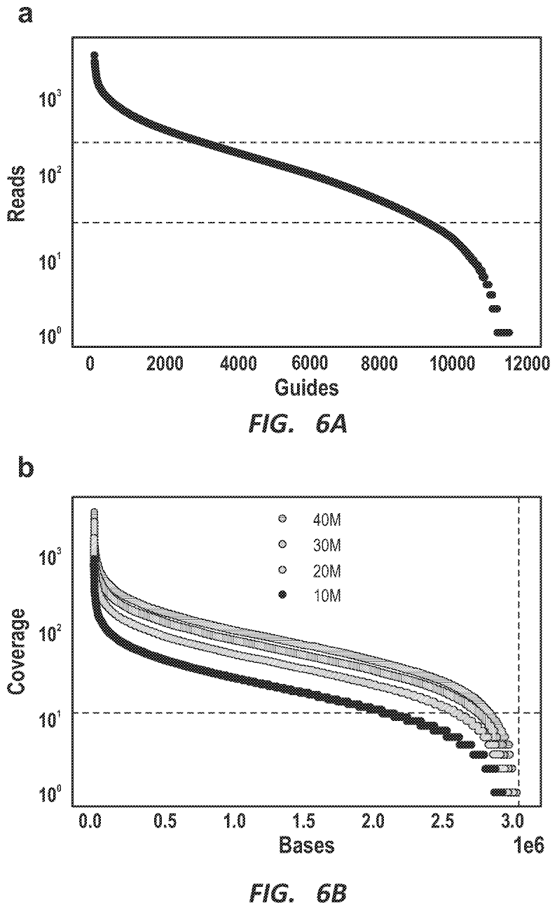

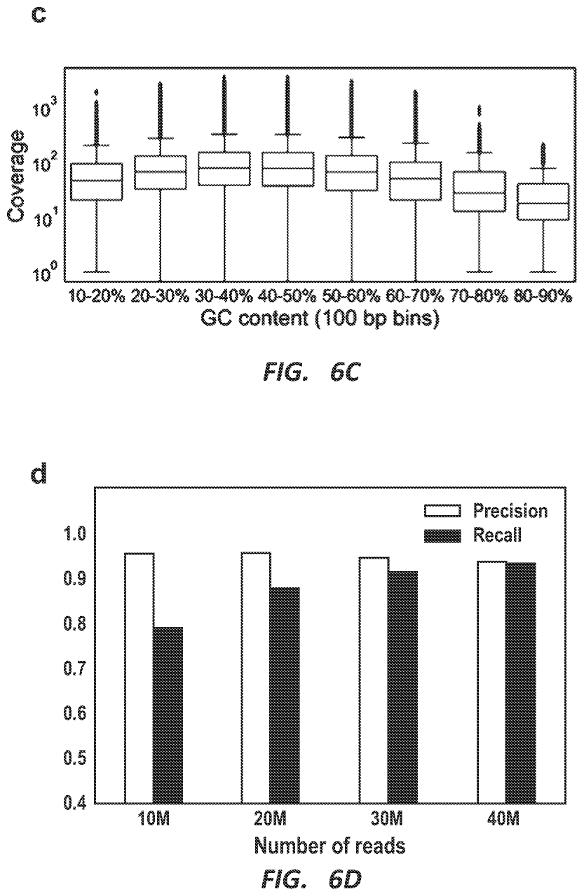

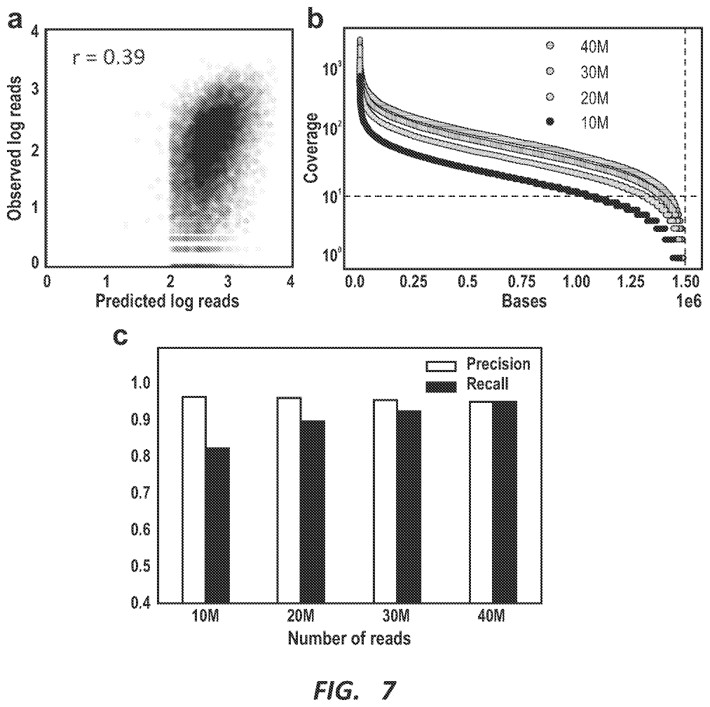

Captures with the optimized guides achieved an average enrichment of 64-fold (6.3% of reads on target) using NA12878 genomic DNA. Guide uniformity improved modestly compared to the naïve guide set (54.0% of guides within one log10 difference, FIG. 6A). While cutting at predicted off target sites was present, it made up a relatively smaller fraction of reads compared to the pilot guides (5.5% of reads). Observed guide performance correlated with predictions (Pearson r=0.38, FIG. 7), but this correlation was lower than the cross-validation results. This may be due to the optimized guides falling within a narrower range of expected performance compared to the cross-validation. For example, when only considering guides above the 2.0 threshold used for picking optimized guides, the cross-validation results in reduced correlation (Pearson r=0.61). Additionally, the pilot guides were subjected to PCR amplification and restriction enzyme digestion steps prior to in vitro transcription while the optimized guides were not. These additional steps may introduce biases that are not present for the optimized guide set.

Raw coverage of the target region was examined at different levels of downsampling. With 20 million read pairs, 84.4% of bases in the target region are covered by at least 10 reads, and increasing to 40 million read pairs covers 92.8% of bases by at least 10 reads (FIG. 6B). Considering only those bases outside of repetitive elements (as defined by Repeat Masker), 20 million read pairs cover 86.7% of bases with at least 10 reads, and at 40 million read pairs 94.6% of bases are covered by at least 10 reads (FIG. 7). Next examined was the GC content coverage bias. 100 basepair bins with extremely low (10-20%) or high (80-90%) GC content have median coverage of 46 and 18, respectively, while the 40-50% bin has median coverage of 78 (FIG. 6C).

### Example 8—Variant Calling

Base quality scores were recalibrated with GATK (version 4.1.2.0, then variants were called with HaplotypeCaller with a minimum base quality score of 20 (−mbq 20). Sample VCFs were compared to the “Platinum” variant calls with hap.py. Single nucleotide variants were considered separately from insertions or deletions.

Finally, single nucleotide variant calling with the downsampled dataset was performed, and it was found that with 20 million read pairs, high precision and recall (0.95 and 0.88, respectively for all bases) compared to Illumina platinum calls for this sample can be achieved. Increasing to 40 million read pairs maintains high precision and boosts recall (0.94 and 0.93, respectively, FIG. 6D). Restricting to bases not Repeat Masked yields slightly improved performance for all conditions (FIG. 7).

### Example 9—Method as Applied to Massively Parallel Sequencing

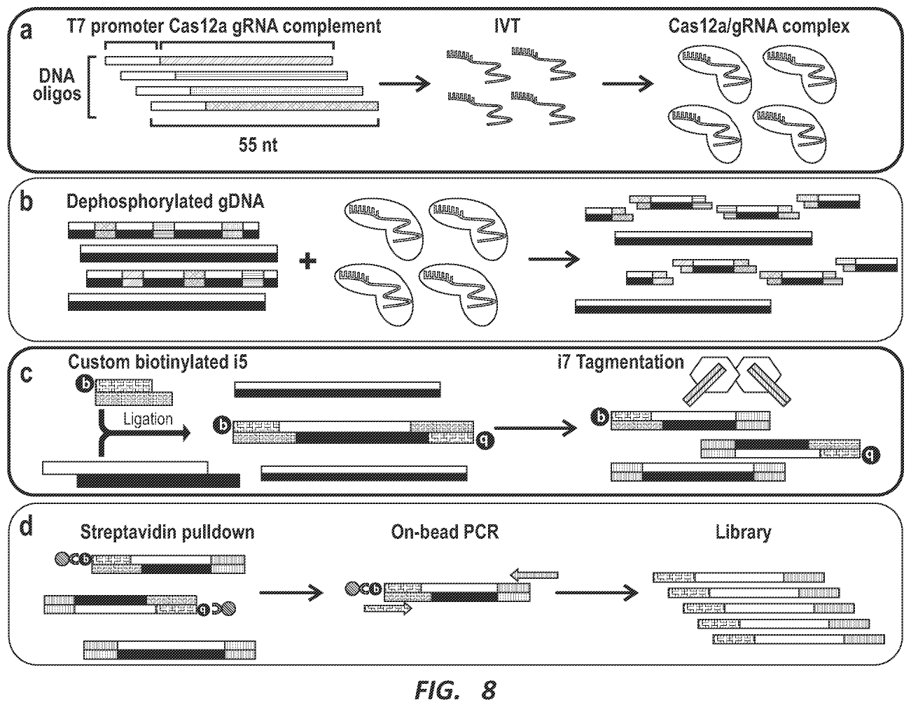

DNA oligonucleotides encoding guideRNA sequences can be synthesized and in vitro transcribed (IVT) into RNAs, which are then complexed with Cas12a in order to make reaction-ready ribonucleoproteins (RNPs) (FIG. 8 panel A). The DNA template can be dephosphorylated (alternatively could be blunted by gap filling and chew back) and then cut with RNPs, yielding sticky ends only at targeted sites (FIG. 8 panel B). One of various custom adapters, with a chemical modification such as biotin (white “b” in black circle) and complementary sticky ends, can be ligated to targets. Then, Tn5 tagmentation can be used to incorporate the second sequencing adapter ubiquitously throughout the DNA template (FIG. 8 panel C). Pulldown of the adapter-ligated targets is achieved with, for example, streptavidin-coated magnetic beads, and then PCR can be performed on the pull-down product to enrich on-target molecules. Standard quantification, size selection, sequencing, and analysis are then performed (FIG. 8 panel D).

It will be apparent to those having skill in the art that many changes may be made to the details of the above-described embodiments without departing from the underlying principles of the invention.

1. A method for targeted sequencing of double-stranded nucleic acids, the method comprising:
cleaving dephosphorylated double-stranded nucleic acids with a plurality of endonuclease-guide ribonucleic acid (gRNA) complexes to generate double-stranded nucleic acid fragments having phosphorylated 5′ end overhangs at targeted sites;
ligating a first adapter to the targeted sites of the double-stranded nucleic acid fragments, wherein the first adapter comprises priming sites;
fragmenting further the double-stranded nucleic acids fragments at random sites;
adding a second adapter at the random sites, wherein the second adapter comprises priming sites; and
amplifying selectively nucleic acid sequences containing the first adapter and the second adapter to generate a library of target sequences.

2. The method of claim 1, wherein each of the plurality of endonuclease-gRNA complexes are ribonucleoproteins.

3. The method of claim 2, wherein each of the plurality of endonuclease-gRNA complexes comprise a CRISPR-Cas12a-based endonuclease complexed with one of a plurality of different gRNA to provide a plurality of different endonuclease-gRNA complexes.

4. The method of claim 3, wherein gRNA is targeted to the target sequence and comprises a protospacer adjacent motif compatible with the CRISPR-Cas12a-based endonuclease.

5. The method of claim 3, further comprising:
synthesizing double-stranded nucleic acids encoding the different gRNA sequences;
transcribing the synthesized double-stranded nucleic acids in vitro into the gRNAs; and
complexing the gRNAs with the CRISPR-Cas12a-based endonuclease to form the plurality of different endonuclease-gRNA complexes.

6. The method of claim 3, further comprising:
complexing commercially-available RNAs with the CRISPR-Cas12a-based endonuclease to form the plurality of different endonuclease-gRNA complexes.

7. The method of claim 1, wherein the double-stranded nucleic acids comprise deoxynucleic acids (DNA), including naturally-occurring DNA, such as genomic DNA, mitochondrial DNA, and cell-free DNA, synthetic DNA, such as complementary DNA (cDNA) (including as reverse transcribed from RNA), and polymerase chain reaction (PCR) products.

8. The method of claim 1, further comprising dephosphorylating double-stranded nucleic acids to provide the dephosphorylated double-stranded nucleic acids.

9. The method of claim 8, further comprising, prior to dephosphorylation, removing existing 5′ end overhangs from double-stranded nucleic acids to provide the double-stranded nucleic acids for dephosphorylation.

10. The method of claim 1, wherein the first adapter comprises double-stranded nucleic acids comprising degenerate overhanging bases compatible with the phosphorylated 5′ end overhangs of the double-stranded nucleic acid fragments.

11. The method of claim 10, wherein the first adapter further comprises a unique molecular identifier, index sequence, or both.

12. The method of claim 11, wherein the first adapter further comprises a 5′ biotin modification compatible with streptavidin pulldown, a digoxigen (DIG) modification compatible with DIG antibody pulldown, a chemical modification compatible with isolation via click chemistry reaction with an alkyne or azide solid resin, or a poly-histidine tag modification compatible with nickel-containing solid resin pulldown.

13. The method of claim 1, further comprising enriching the double-stranded nucleic acid fragments containing the first adapter ligated thereto, preceding or after fragmenting further the double-stranded nucleic acids.

14. The method of claim 1, wherein fragmenting further the double-stranded nucleic acids fragments at random sites and adding the second adapter at the random sites is accomplished in a single step or in two or more steps.

15. The method of claim 1, wherein fragmenting further the double-stranded nucleic acids fragments at random sites and adding the second adapter comprises using a transposase with a commercially-available or custom adapter.

16. The method of claim 1, wherein fragmenting further the double-stranded nucleic acids fragments at random sites comprises enzymatic fragmentation, sonic fragmentation, or mechanical sheering.

17. The method of claim 1, wherein amplifying selectively nucleic acid sequences containing the first adapter and the second adapter to generate a library of target sequences.

18. The method of claim 1, further comprising performing library quantification techniques, size selection, massively parallel sequencing, informatic protocols, or combinations thereof, to the library of target sequences.

19. The method of claim 1, wherein the target sequence comprises whole genes, a region of interest, or a list of regions of interest.

20. The method of claim 1, wherein the target sequences comprise regions of high or low guanine-cytosine (GC) content.

21. The method of claim 1, further comprising generating the library of target sequences without a size selection step prior to addition of the first and second adapters.

22. A method of designing a pool of guide RNA (gRNA) to be complexed with an endonuclease, the method comprising:
identifying all possible target sites of the endonuclease within target sequences;
providing a first plurality of gRNA to target each of the identified possible target sites of the endonuclease;
complexing each of the first plurality of gRNAs with the endonuclease to form a first plurality of endonuclease-gRNA complexes;
performing the steps of claim 1 utilizing the first plurality of endonuclease-gRNA complexes to generate a first library of the target sequences;
comparing the first library of the target sequences to a known library of the target sequences;
determining a subset of the first plurality of endonuclease-gRNA complexes that generate target sequences aligned with the known library of the target sequences;
determining molecular features of the target sequences associated with the subset of the first plurality of endonuclease-gRNA complexes; and
designing a second plurality of gRNA to the same or additional target sequences that also have the molecular features associated with performance of the subset of the first plurality of endonuclease-gRNA complexes.

23. A first sequencing adapter mixture comprising:
a plurality of double-stranded nucleic acids each having a first strand and a second strand, wherein each first strand comprises priming sites; wherein each second strand is complementary to the respective first strand, forms a double-stranded region with the first strand, and contains a 5′ overhang of one, two, three, four or five degenerate bases.

24. The first sequencing adapter of claim 23, wherein the first strand comprises a unique molecular identifier relative to the other first strands in the mixture, an index sequence, or both.

25. The first sequencing adapter of claim 24, wherein the unique molecular identifier, index sequence, or both is towards the 5′ end of the first strand when compared to sequences complementary to the respective second strand.

26. The first sequencing adapter of claim 23, wherein the first sequencing adapter further comprises a 5′ biotin modification compatible with streptavidin pulldown, a digoxigen (DIG) modification compatible with DIG antibody pulldown, a chemical modification compatible with isolation via click chemistry reaction with an alkyne or azide solid resin, or a poly-histidine tag modification compatible with nickel-containing solid resin pulldown.
